(19) United States
(12) Patent Application Publication
(10) Pub. No.: US 2002/0034731 A1
(43) Pub. Date: Mar. 21, 2002

(54) Method and apparatus for measuring characteristics by analyzing a sample of human or animal body substances

(76) Inventors: Marcel Thuerk, Bovenden (DE); Ruediger Goetz, Goettingen (DE); Joerg Wichard, Goettingen (DE); Christian Merkwirth, Goettingen (DE); Fabian Evert, Rosdorf (DE); Stefan Kamphausen, Reinhausen (DE); Claus Brunzema, Goettingen (DE); Martin Wiesenfeldt, Sulzbach (DE)

Correspondence Address: CROWELL & MORING LLP, INTELLECTUAL PROPERTY GROUP, P.O. BOX 14300, WASHINGTON, DC, 20044-4300, US

(21) Appl. No.: 09/942,040

(22) Filed: Aug. 30, 2001

(30) Foreign Application Priority Data
Aug. 30, 2000 (DE) 100 42 847.9

(51) Int. Cl. C12Q 1/00; C12Q 1/02

(52) U.S. Cl. 435/4; 435/29

(57) ABSTRACT

A method and apparatus (6) for measuring characteristics by analyzing a sample (1) of human or animal body substances, particularly blood or tissue. To this end, a known separation procedure is first used to distribute the sample (1) on a substrate (7). Subsequently, the sample (1) is divided into individual segments (8) and in the subsequent analysis is examined in an unchanged position on the substrate (7). The selection of a suitable procedure for analyzing a respective sample and of the segments (8) to be analyzed is determined as a function of the measured characteristics of previous segments (8). This makes it possible as the process is carried out to specify the characteristics to be measured which are decisive for an ultimate diagnosis, so that the result is obtained much more quickly. In addition, the minimal dimensions of the apparatus (6) make it suitable also for mobile use on site.

The invention relates to a method for measuring characteristics by analyzing a sample of a human or animal body substance, particularly blood or tissue, using a separation procedure on a substrate to prepare a subsequent analysis of the starting products obtained in the separation procedure. The invention further relates to an apparatus for carrying out this method.

In practice, such a method is used in medicine to detect the characteristics of human or animal blood, tissue or other specimens. The separation process used, in particular, is a two-dimensional electrophoresis procedure for separating cellular constituents. The constituents are distributed on the substrate and removed incrementally for analysis.

Such a device is furthermore known, for instance, from U.S. Pat. No. 6,065,754 in which a robot removes the individual samples from the electrophoresis surface and transfers them for subsequent analysis.

Furthermore, U.S. Pat. No. 5,245,185 discloses another device in which the sample can be transferred from the electrophoresis surface for subsequent examination by laser ionization.

In practice, the disadvantage of both of these methods is the considerable complexity required to remove the sample, which is distributed on the substrate by the separation procedure, and to transfer it for subsequent analysis. In particular, handling aids are required, which can essentially be achieved only under laboratory conditions or in a stationary environment. The known procedures are therefore not suitable for mobile use, e.g., at the site of an accident. In practice, this causes valuable time to be lost, which is often not available in emergencies, particularly for a reliable diagnosis.

### SUMMARY OF THE INVENTION

Thus, one object of the invention is to provide a method for measuring characteristics of a body substance, which method is simple to handle and suitable not only for stationary applications but also for use on site in the field.

A further object of the invention is to provide an apparatus for carrying out the method of the invention.

The first-mentioned object is attained according to the invention by providing a method for measuring characteristics by analyzing a sample of human or animal body substances comprising separating the sample using a separation procedure carried out on a substrate in preparation for subsequent analysis; dividing the substrate bearing the separated sample into individual segments, and analyzing the sample in a plurality of different segments on the substrate.

The further object is achieved by providing an apparatus for measuring characteristics by analyzing a sample of a human or animal body substance, in which the apparatus comprises a substrate that carries the body substance, and the apparatus measures characteristics of the body substance distributed over the substrate on spatially resolved segments of the substrate.

Further details and preferred aspects of the invention are set forth in the following description.

Thus, according to the invention, a method is provided in which the sample is distributed over individual segments of a substrate on which it is supported, and a specific analysis of the body substances of different segments is carried out on the substrate. Since the body substances are analyzed directly on the substrate, the complexity required to handle the sample in the prior art is eliminated. Thus, the method according to the invention is also particularly suitable for mobile use at the site of an accident, since especially mechanical aids, such as robot arms, can be dispensed with. At the same time, accuracy in the measurement of the characteristics can be significantly increased since the sample can remain on the substrate and, as a result, is essentially not subject to any additional external influences after performance of the separation procedure. Measured value deviations caused by removal of the sample from the substrate are thus excluded.

A particularly advantageous embodiment of the present invention is achieved by using the measured characteristics of a previous segment of the sample to determine the analysis of a subsequent segment. Consequently, the significant values of the previous sample and the characteristics that can be derived therefrom already create the conditions for the analysis of the subsequent segment, so that the result is obtained faster and with greater precision and reliability. In contrast to the methods known in the prior art, in which the analysis sequence of the different segments is determined in advance and is essentially unalterable, this method achieves a dynamic adaptation of the test sequence in which the individual results of the different segments are related to each other. In this manner, the amount of data can be reduced in that recognizable logical interrelationships between the analysis results of different segments are used for evaluation, and characteristics that are not foreseeably useful therefore subsequently no longer need to be taken into consideration. This reduces the time required for carrying out the method, i.e., the analysis time.

In one particularly advantageous embodiment of the invention, the measured characteristics of the preceding segment are used to determine the analytical procedure. As a result, the analytical procedure to be performed following the separation procedure can already be influenced after the first measurement of characteristics. Consequently, a procedure that may be inappropriate in a particular case will be recognized as such, and a suitable procedure will be determined by means of known expert systems. Any inadequate measurement of characteristics due to use of an inappropriate technique can thus be recognized in time, and this information can be used directly to devise or select a suitable test procedure while a specified measurement series is underway.

It is also particularly advantageous if the results of the analysis of the preceding segment are used to select the segment to be analyzed next. Consequently, those segments of the sample that are of little use can be identified based on the previous segments, so that measured value acquisition can be specifically tailored to appropriate subsequent segments. Both the adjacent segments and non-adjacent segments can be highly significant for measured value acquisition, which in each individual case can be determined from the nature and magnitude of the measured characteristics.

A further and particularly practical modification of the method according to the invention is achieved by using the analysis to perform an initial rough classification in order to limit the characteristics that subsequently must be measured. The characteristics to be measured are therefore selected based on a higher level classification to avoid measuring unnecessary characteristics. This will substantially decrease the computation effort as well as the corresponding time required.

It is also particularly advantageous if a plurality of analytical procedures are performed successively and their selection or sequence is determined by the result of at least one preceding analytical procedure. In this way the respectively suitable analytical procedure can be adapted to the preceding results, and this, in particular, leads more rapidly to a precise result and therefore to an earlier diagnosis. Recourse to previously analyzed segments is feasible, where necessary or desired. Likewise, it is feasible to determine the characteristics of the sample by means of an additional analytical procedure for control purposes.

A particularly advantageous further development of the invention is achieved by using the sequence of the analysis of the different segments as an additional basis for measurement. Thus, the reproducible sequence of the analysis can provide important information for the subsequent diagnosis, which can be of decisive importance for the final evaluation of the results.

The second-mentioned object is to provide an apparatus having a substrate that supports the body substances for carrying out the method for measuring characteristics of a body substance by analyzing a sample of a human or animal body substance, particularly blood or tissue. This object is attained according to the invention in that the apparatus is constructed for spatially resolved acquisition of measured values of the body substance supported on the substrate. As a result, the sample can remain on the substrate for analysis. This eliminates additional handling and simultaneously reduces or excludes environmental influences that can adversely affect the result. The dimensions of the device can be minimized such that it is readily portable for mobile use on site. The device is furthermore less costly to produce.

Particularly advantageous is an embodiment of the device according to the invention, in which the device is designed for optical detection of the characteristics to be measured by means of a light source and an optical sensor. This makes it possible to conduct the analysis without any direct contact with the sample, so that external influences can be kept very low. Various wavelength ranges from the infrared to the visible to the ultraviolet spectrum can be used for the light. The light beam emitted by the light source, after suitable focussing, strikes the sample, which is thus especially transilluminated. Variations in the characteristic values of the light are detected by the optical sensor.

For this purpose, the light source can be arranged so as to be movable. It is particularly advantageous, however, if the device for the analysis of different segments of the sample is equipped with controllable light deflection. This makes it possible for the light source to be fixed and stationary and for the light to be directed onto certain segments of the sample by light deflection, e.g., by means of a mirror and by focussing.

For this purpose, a particularly advantageous modification requires the substrate to be transmissive or light permeable. The optical sensor for detecting the light passing through the sample can thus be arranged directly adjacent a side of the substrate that faces away from the sample. This reduces susceptibility to disturbances and requires little space.

In another particularly advantageous modification, the apparatus for analysis of different segments of the sample is equipped with a relatively movable substrate. In particular, the substrate or base can be constructed so as to be movable in relation to the other components of the device, so that any segments of the sample supported on the substrate can be analyzed in detail.

Another particularly advantageous embodiment is provided if the device is equipped with an ultrasound source and an ultrasound sensor. As a result, ultrasound tests can be conducted in addition to or in lieu of an optical analysis. This further improves the reliability of the diagnosis established therefrom.

It is also particularly practical if the apparatus additionally has a sensor that can be associated with a respective segment of the substrate. This sensor can be associated with the segment to be examined either continuously or as needed, so that the required measurements are taken only in the desired segment. Any influence of adjacent segments can thus be excluded.

It is furthermore particularly advantageous if the additional sensor is designed to detect the electrical conductivity or the pH value of the sample in the test segment to determine additional characteristics of the body substance by means of which the reliability of the analysis can be further increased.

### BRIEF DESCRIPTION OF THE DRAWINGS

The invention is susceptible to different variations. To illustrate the basic principle, one of these will now be described in greater detail with reference to the drawing in which

FIG. 1 is a flow diagram of the method according to the invention and

FIG. 2 is a schematic side view of an apparatus for carrying out the method depicted in FIG. 1.

### DETAILED DESCRIPTION OF PREFERRED EMBODIMENTS

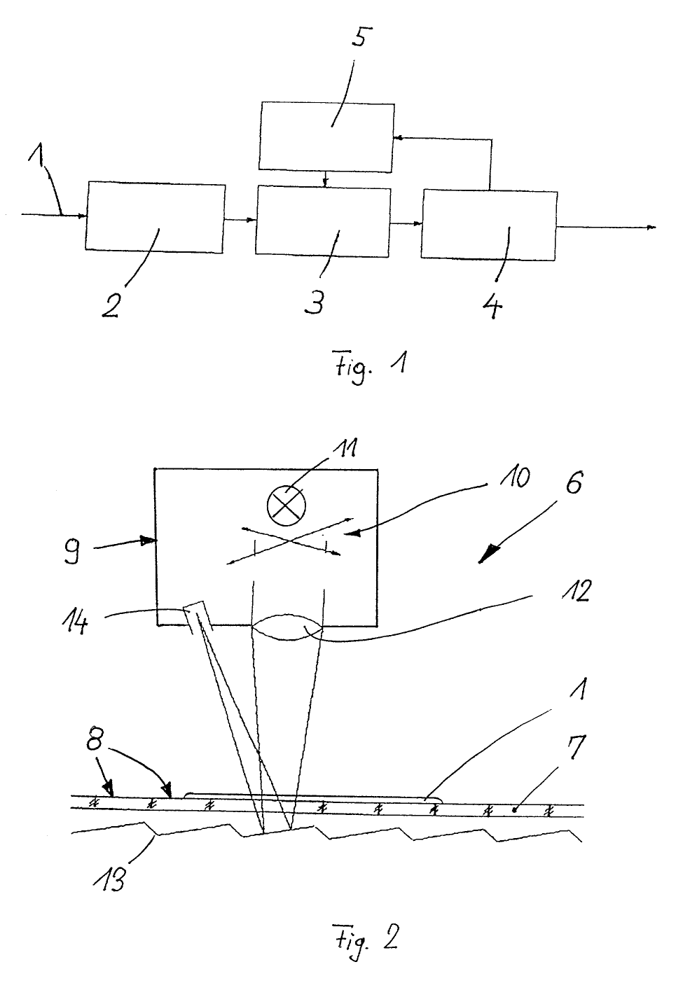

FIG. 1 shows a flow diagram of the method according to the invention. A sample 1, which is shown in greater detail in FIG. 2, is supplied for analysis, which in a first process step comprises an electrophoresis separation procedure 2. A subsequent analysis procedure 3 is carried out by means of a device 6, shown in greater detail in FIG. 2. This analysis procedure supplies the necessary characteristics for a rough classification of sample 1. Based on this first analysis, a number of abnormal functions in the body of the patient may be suspected, for example. Thus, the initial rough classification leads to selection or creation of a test procedure resulting from the initial analysis of the characteristics. This analysis of the characteristics is received by an evaluation unit 4 and then transmitted to a control unit 5. The control unit then modifies the process to be carried out in the subsequent analysis steps depending on the previously measured characteristics. This modification can be mechanical, electrical or logical in nature. For instance, if in the initial rough classification additional information is provided by an IR spectrum in the near infrared region, this modification could, for example, consist of increasing the resolution within a certain wavelength window or measuring a different wavelength range, or using the pulse response to an ultrasound pulse. This reconfiguration of the test procedure may be iterated repeatedly until the diagnosis for the diagnostic point or points to be examined is completed. Subsequently, the analytical procedures not previously used can be performed.

FIG. 2 is a schematic sketch of an apparatus 6 by means of which the analytical procedure can be performed. Sample 1 as the starting product of the preceding separation procedure lies on a two-dimensional substrate 7, which preferably comprises an electrophoresis plate, and which is divided into individual segments 8. To measure optical characteristics, device 6 is equipped with an optical unit 9, which in the illustrated embodiment can be moved relative to the substrate 7 by means of a displacement unit 10. A light beam 11 emitted by a light source is first focussed through a lens 12 and subsequently strikes sample 1. Sample 1 is at least partially transilluminated, so that the light beam initially passes through the substrate 7, which is transmissive for this purpose, and strikes a reflection unit 13 on the side of substrate 7 facing away from the optical unit 9. The reflection unit directs the light beam through sample 1 again and thence to an optical sensor 14 embodied as a photodetector. For the light, various wavelength ranges can be used, from the far infrared all the way to the ultraviolet spectral range.

The foregoing description and examples have been set forth merely to illustrate the invention and are not intended to be limiting. Since modifications of the described embodiments incorporating the spirit and substance of the invention may occur to persons skilled in the art, the scope of the invention should be construed broadly to include all variations within the scope of the appended claims and equivalents.

What is claimed is:

1. A method for measuring characteristics by analyzing a sample of human or animal body substances comprising: separating the sample using a separation procedure carried out on a substrate in preparation for subsequent analysis; dividing the substrate bearing the separated sample into individual segments, and analyzing the sample in a plurality of different segments on the substrate.

2. A method according to claim 1, wherein the analyzed sample is blood or tissue.

3. A method as claimed in claim 1, wherein the analysis of a subsequent segment is determined by the measured characteristics of a preceding segment of the sample.

4. A method as claimed in claim 1, wherein measured characteristics of at least one previously analyzed segment are used to determine the procedure followed to analyze at least one subsequent segment.

5. A method as claimed in claim 1, wherein a result of analysis of at least one previously analyzed segment is used to select the segment that is to be analyzed next.

6. A method as claimed in claim 1, further comprising carrying out a rough classification of the sample based on initial analysis results, and limiting the characteristics to be analyzed in subsequently analyzed segments based on the rough classification of the sample.

7. A method as claimed in claim 1, wherein a plurality of analytical procedures are carried out in sequence, and the sequence of the analytical procedures is selected based on a result of analysis of at least one previously analyzed segment.

8. A method as claimed in claim 1, wherein a plurality of analytical procedures are carried out successively, and the analytical procedures to be carried out are selected based on an analysis result of at least one previously analyzed segment.

9. A method as claimed in claim 1, wherein the sequence of the analysis of the different segments is used as a basis for measurement acquisition.

10. Apparatus for measuring characteristics by analyzing a sample of a human or animal body substance, said apparatus comprising a substrate that carries the body substance, wherein said apparatus measures characteristics of the body substance distributed over the substrate on spatially resolved segments of the substrate.

11. An apparatus as claimed in claim 10, wherein said body substance is blood or tissue.

12. An apparatus as claimed in claim 10, wherein said apparatus comprises a light source for illuminating the segments of the substrate carrying the body substance sample, and an optical sensor for sensing light reflected or transmitted from respective segments of the substrate to optically measure characteristics of the body substance sample.

13. An apparatus as claimed in claim 12, wherein said light source is provided with means for controllably deflecting light from the light source to respective segments of the substrate.

14. An apparatus as claimed in claim 10, wherein said substrate is transmissive to light.

15. An apparatus as claimed in claim 10, wherein said substrate is movable relative to a measurement device so that the measurement device can measure characteristics of the body substance sample distributed on different spatially resolved segments of the substrate.

16. An apparatus as claimed in claim 15, wherein said measurement device comprises a light source for illuminating respective spatially resolved segments of the substrate carrying the body substance sample, and an optical sensor for sensing light reflected or transmitted from the respective segments of the substrate to optically measure characteristics of the body substance sample.

17. An apparatus as claimed in claim 16, wherein said apparatus further comprises an ultrasound source for applying ultrasonic energy to the segments of the substrate carrying the body substance sample, and an ultrasound sensor for sensing ultrasonic energy reflected or transmitted from respective segments of the substrate to acoustically optically measure characteristics of the body substance sample.

18. An apparatus as claimed in claim 10, wherein said apparatus comprises an ultrasound source for applying ultrasonic energy to the segments of the substrate carrying the body substance sample, and an ultrasound sensor for sensing ultrasonic energy reflected or transmitted from respective segments of the substrate to acoustically optically measure characteristics of the body substance sample.

19. An apparatus as claimed in claim 10, wherein said apparatus comprises at least one sensor associated with respective segments of the substrate.

20. An apparatus as claimed in claim 19, wherein said sensor senses a characteristic of said body substance sample selected from the group consisting of electrical conductivity and pH value.
